SEQUENCE LISTING (30 sequences)

SEQ ID NO: 1 - DNA, 24 bp, Mus musculus
gtcaatcctacccccgaagcagct

SEQ ID NO: 2 - DNA, 20 bp, Mus musculus
cagcatgggctccttctcca

SEQ ID NO: 3 - DNA, 20 bp, Mus musculus
atggatctcctgaaggtgct

SEQ ID NO: 4 - DNA, 20 bp, Mus musculus
aagagggcagatctatcgga

SEQ ID NO: 5 - DNA, 24 bp, Mus musculus
gacaactacctctcagattatggc

SEQ ID NO: 6 - DNA, 18 bp, Mus musculus
cgggagcatgggaaaagc

SEQ ID NO: 7 - DNA, 17 bp, Mus musculus
catgaagagagctcacc

SEQ ID NO: 8 - DNA, 17 bp, Mus musculus
aatgtgtccgaggccac

SEQ ID NO: 9 - DNA, 18 bp, Mus musculus
atggacccatcgatgtgg

SEQ ID NO: 10 - DNA, 17 bp, Mus musculus
aaaggccagggcactgg

SEQ ID NO: 11 - DNA, 20 bp, Mus musculus
ctcgaagtgttggatacagg

SEQ ID NO: 12 - DNA, 20 bp, Mus musculus
tggcctataggctcatagtg

SEQ ID NO: 13 - DNA, 22 bp, Mus musculus
ctgctgcagaggttattcctcg

SEQ ID NO: 14 - DNA, 24 bp, Mus musculus
ggaagagtgagcggcgcatcaagg

SEQ ID NO: 15 - DNA, 24 bp, Mus musculus
tgcaaaggctccaggtctgagggc

SEQ ID NO: 16 - DNA, 24 bp, Mus musculus
gccaacaccaacctgtccaagttc

SEQ ID NO: 17 - DNA, 20 bp, Mus musculus
ctcagtccttctcttctccg

SEQ ID NO: 18 - DNA, 20 bp, Mus musculus
tgttcctcacgatgtttggg

SEQ ID NO: 19 - DNA, 28 bp, Mus musculus
gcagaaggcagtggagctggacaaagcc

SEQ ID NO: 20 - DNA, 30 bp, Mus musculus
ttgcacttgtagcgacggttctggaaccag

SEQ ID NO: 21 - DNA, 20 bp, Mus musculus
gccctgagtctgaggacaag

SEQ ID NO: 22 - DNA, 19 bp, Mus musculus
atcgtgttcttgctgccag

SEQ ID NO: 23 - DNA, 21 bp, Mus musculus
cactatgggcacagcagctcc

SEQ ID NO: 24 - DNA, 21 bp, Mus musculus
ttgcagctggcctgcgatgtc

SEQ ID NO: 25 - DNA, 17 bp, Mus musculus
gcttcaccaccttcttg

SEQ ID NO: 26 - DNA, 17 bp, Mus musculus
tcaccatcttccaggag

SEQ ID NO: 27 - DNA, 27 bp, Mus musculus
cttttaaagttatcagctcagcgaagc

SEQ ID NO: 28 - DNA, 27 bp, Mus musculus
gcttcgctggactgataactttaaaag

SEQ ID NO: 29 - DNA, 27 bp, Mus musculus
cttttaaagttaagagctcagcgaagc

SEQ ID NO: 30 - DNA, 27 bp, Mus musculus
gcttcgctggactcttaactttaaaag

(19) United States
(12) Patent Application Publication
(10) Pub. No.: US 2009/0054500 A1
(43) Pub. Date: Feb. 26, 2009

(54) Composition and methods useful in preventing cardiac hypertrophy

(75) Inventor: Michael Chin, Needham, MA (US)

Correspondence Address: LAW OFFICE OF MICHAEL A. SANZO, LLC, 15400 CALHOUN DR., SUITE 125, ROCKVILLE, MD, 20855, US

(73) Assignee: The Brigham and Women's Hospital, Inc., Boston, MA (US)

(21) Appl. No.: 11/919,846

(22) PCT Filed: May 12, 2006
(86) PCT No.: PCT/US06/18640

§ 371 Date: Feb. 8, 2008

Related U.S. Application Data
(60) Provisional application No. 60/680,038, filed on May 12, 2005.

(51) Int. Cl. A61K 31/4409; A61P 9/00; C07D 211/70; C12Q 1/68; C12N 5/00

(52) U.S. Cl. 514/357; 514/277; 546/334; 546/342; 435/6; 435/375

(57) ABSTRACT

The present invention is directed to compositions and methods that can be used for preventing the hypertrophy of cardiac cells. The compositions and methods involve the use of compounds that lead to an increase in the cellular activity of the transcription factor CHF1.

### CROSS REFERENCE TO RELATED APPLICATIONS

The present application claims priority to and the benefit of U.S. provisional application 60/680,038, filed on May 12, 2005.

### STATEMENT OF GOVERNMENT FUNDING

The United States Government has a paid-up license in this invention and the right in limited circumstances to require the patent owner to license others under reasonable terms as provided for by the terms of NIH Grant No. HL081088

### FIELD OF THE INVENTION

The present invention is directed to compounds that can be administered to subjects to inhibit the hypertrophy of cardiomyocytes. Such compounds are useful in the treatment or prevention of diseases such as congestive heart failure. The invention also includes methods of determining if a test compound is useful in the treatment of a condition characterized by cardiomyocyte hypertrophy by assaying the compound for its ability to suppress a phenotypic characteristic caused by a loss of CHF1 expression.

### BACKGROUND OF THE INVENTION

Congestive heart failure (CHF) is a common condition, affecting approximately 5 million people in the United States. Although CHF can be treated using beta blockers and ACE inhibitors, the overall prognosis is poor. Only about 50% of patients survive for five years from the time of diagnosis and only about 20% survive ten years. Pathologically, it appears that CHF is often caused by hypertrophy of cardiomyocytes which leads to a thickening of ventricular walls.

The hairy-related basic helix-loop-helix (bHLH) transcriptional repressor CHF1 (also called Hey2, Hesr-2, Hrt2, HERP1, and gridlock) is thought to play an important role in cardiovascular development ((Chin, et al., J. Biol. Chem. 275: 6381-6387 (2000); Iso, et al., Mol. Cell Biol. 21:6071-6079 (2001); Kokubo, et al., Biochem Biophys Res Commun 260:459-465 (1999); Leimeister, et al., Mech. Dev. 85:173-177 (1999); Nakagawa, et al., Dev. Biol. 216:72-84 (1999); Zhong, et al., Science 287:1820-1824 (2000); Iso, et al., J. Cell Physiol. 194:237-255 (2003); Fischer, et al., Trends Cardiovasc Med. 13:221-226 (2003)). Targeted disruption of CHF1/Hey2 in mice leads to congenital heart abnormalities and cardiomyopathy (Donovan, et al., Curr. Biol. 12:1605-1610 (2002); Gessler, et al., Curr. Biol. 12:1601-1604 (2002); Kokubo, et al., Circ. Res. 95: 540-547 (2004); Sakata, et al., Proc. Nat'l Acad. Sci. USA 99:16197-16202 (2002)). In one line of knockout mice, the development of cardiomyopathy in adults appeared to be independent of congenital anomalies (Sakata, et al., Proc. Nat'l Acad. Sci. USA 99:16197-16202 (2002)). This finding indicates that CHF1-dependent transcriptional programs are important in maintenance of normal ventricular function.

GS4012 (4-(2-(4-Methoxyphenylsulfanyl)ethyl)pyridine, HCl) is a cell-permeable compound that suppresses the gridlock phenotype associated with mutations in the CHF1 gene in zebrafish (Peterson, et al., Nat. Biotechnol. 22:595-599 (2004); see also Sakata, et al., Proc. Nat'l Acad. Sci. USA 99:16197-16202 (2002)). As such, GS4012 and compounds structurally related to GS4012 should be useful in treating or preventing other diseases or conditions resulting from mutations in this gene.

### SUMMARY OF THE INVENTION

The present invention is based upon the discovery that mice lacking the transcription factor CHF1 develop heart failure and a thin-walled myocardium. In contrast, mice that overexpress this factor, and cardiac cells isolated from these mice, are resistant to agents that induce hypertrophy. This has lead to the concept that agents that increase CHF1 activity should be useful in treating diseases characterized by the excessive hypertrophy of cardiac cells, e.g., congestive heart failure or familial cardiac hypertrophy. This concept was further supported by experiments performed using GS4012. When this compound was tested on cardiomyocytes in vitro, it was found to prevent phenylephrine-induced hypertrophy, i.e., it replicated the effect of CHF1 overexpression. Thus, GS4012 and structurally similar compounds should be useful as therapeutic agents.

In its first aspect, the invention is directed to a pharmaceutical composition in unit dose form that contains a compound increasing the activity of the CHF1 gene product in cardiomyocytes and a pharmaceutically acceptable vehicle. The vehicle may be water, saline, or other fluid used to dissolve or suspend the compound or, in the case of solid dosage forms, the vehicle may be a pharmaceutically inert solid composition, e.g., a powder or granular composition, in which the compound is mixed or suspended. Pharmaceutical compositions may also include one or more excipients, e.g., a flavoring agent, dispersing agent, etc. The term “unit dose form” as used herein refers to a single entity for drug administration, such as a tablet or capsule. The term “CHF1 gene product” refers to either the human protein as shown in GenBank accession no. AF173901, or the mouse sequence as shown in GenBank accession no. AF173902. Preferably the compound used in pharmaceutical compositions is GS4012 (4-(2-(4-methoxyphenylsulfanyl)ethyl)pyridine, HCl) or a GS4012 derivative. These compounds are defined by formula I:

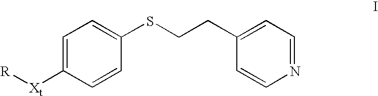

where X is O, N, C or Sand t is 0 or 1
R is selected from the group consisting of: H; a straight or branched C₁-C₆ alkyl; a straight or branched C₂-C₆ alkenyl; —CH₂)ₐ—C(O)—R₁; and —CH₂)ₐ—NR₂R₃ where R₁-R₃ are each independently selected from: H; halogen; and C₁-C₃ alkyl; and a is an integer from 0 to 3.
and wherein the phenyl or pyridine rings may optionally each independently be substituted by one or more substituents selected from the group consisting of: a halogen; OH; and C₁-C₃ alkyl.

When R is methyl, the structure of formula I is that of GS4012. Although this compound is known in the art, other derivatives defined above are not and are part of the invention as compounds per se. These derivatives should preferably be in an isolated form, i.e., they should be essentially free of other active ingredients and would typically constitute at least 85% by weight of the total active compounds in a preparation. The term “active compounds” means compounds other than solvents, excipients and the like that are used for the purpose of dissolving compounds, or maintaining their stability. Derivatives of particular interest occur when R is a straight or branched C₁-C₃ alkyl; —(CH₂)ₐ—C(O)—R₁; or —(CH₂)ₐ—NR₂R₃; where R₁-R₃ are independently H or OH and a is 1 or 2. The most preferred derivatives are: 4-[2-(4-pyridinyl)-ethylsulfanyl]phenoxy acetic acid (shown as structure II) and 4-[2-(4-dimethylaminoethoxy-phenylsulfanyl)-ethyl]pyridine (shown as structure III):

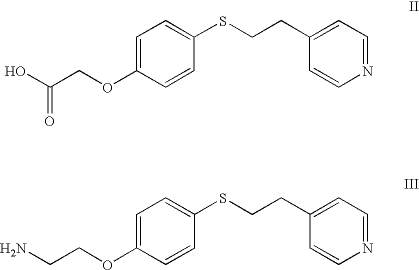

The invention also includes methods of preventing the hypertrophy of cardiomyocytes either in vitro or in vivo by administering a factor increasing CHF1 gene expression. For example, cells might be transfected with an expression vector encoding human CHF1 or a patient might be administered a compound that increases CHF1 activity such as the compounds described above. In vitro studies will be of value to scientists studying diseases characterized by cardiac cell hypertrophy such as congestive heart failure or familial cardiac hypertrophy. In vivo administration of compounds increasing the activity of CHF1 may also be used by scientists studying these diseases and may also be used therapeutically as a treatment procedure.

In another aspect, the invention is directed to a method of determining if a test compound is useful in preventing cardiac hypertrophy. This can be accomplished by first assaying the test compound for its ability to suppress some other phenotypic characteristic caused by a loss of CHF1 expression. The objective of this first assay is to rapidly screen a large number of compounds. For example, rapid screening for compounds altering mutations in CHF1 may be accomplished using the gridlock phenotype in zebrafish (see Peterson, et al., Nat. Biotechnol. 22:595-599 (2004)). Once compounds that are active in suppressing the phenotype have been identified, they may be further examined in a second assay to confirm an effect on cardiomyocyte hypertrophy. For example, cardiac myocytes may be isolated and grown in vitro in the presence of a compound that stimulates hypertrophy, e.g., phenylephrine. By comparing growth in the presence and absence of a compound identified in the initial screening assay, it can be determined whether the compound inhibits cellular hypertrophy. Compounds that are identified as being active have potential use as therapeutic agents in diseases such as congestive heart failure and familial cardiac hypertrophy.

### DETAILED DESCRIPTION OF THE INVENTION

A. GS4012 and Other Related Compounds

GS4012 (4-(2-(4-methoxyphenylsulfanyl)ethyl)pyridine, HCl) is a cell-permeable pyridinyl-thioether compound that induces VEGF and VEGF-mediated vessel formation (Peterson, et al., Nat. Biotechnol. 22:595 (2004)). It can be purchased commercially (available from Merck and CalBiochem) or made using standard methods well known in the art. Changes may be made to the compound to produce the derivatives described above using standard methodology. It will be understood that, unless otherwise indicated, reference to GS4012 and the derivatives of GS4012 described herein includes any biologically acceptable salt or acid form of these compounds.

B. Route of Administration

The methods and compositions discussed above are compatible with any dosage form or route of administration. Thus, agents may be administered orally, intranasally, rectally, sublingually, buccally, parenterally, or transdermally. Dosage forms may include tablets, trochees, capsules, caplets, dragees, lozenges, parenterals, liquids, powders, and formulations designed for implantation or administration to the surface of the skin. The most preferred dosage forms are tablets or capsules for oral administration, typically containing 50 μg-500 mg of active compound. All dosage forms may be prepared using methods that are standard in the art (see e.g., Remington's Pharmaceutical Sciences, 16th ed. A. Oslo. ed., Easton, Pa. (1980)).

Active ingredients may be used in conjunction with any of the vehicles and excipients commonly employed in pharmaceutical compositions, e.g., talc, gum arabic, lactose, starch, magnesium stearate, cocoa butter, aqueous or non-aqueous solvents, oils, paraffin derivatives, glycols, etc. Coloring and flavoring agents may also be added to preparations designed for oral administration. Solutions can be prepared using water or physiologically compatible organic solvents such as ethanol, 1-2 propylene glycol, polyglycols, dimethylsulfoxide, fatty alcohols, triglycerides, partial esters of glycerin, and the like. Parenteral compositions containing active ingredients may be prepared using conventional techniques and include sterile isotonic saline, water, 1,3-butanediol, ethanol, 1,2-propylene glycol, polyglycols mixed with water, Ringer's solution, etc.

C. Treatment Methods

The compounds described herein are especially useful in the treatment or prevention of diseases or conditions resulting from the hypertrophy of cardiomyocytes, e.g., congestive heart failure, e.g., by administering GS4012 or similar compounds. It is expected that the skilled practitioner will adjust the dosage of compounds on a case by case basis using methods well established in clinical medicine. The daily dose, e.g., 100 μg-1 g, will be influenced by factors such as the age of the patient, the disease state, side effects associated with the particular agent being administered and other clinically relevant factors. In some cases, a patient may already be taking medications at the time that treatment with the present compound is initiated. These other medications may be continued provided that no unacceptable adverse side effects are reported by the patient. Daily dosages may be provided in either a single or multiple regimen with the latter being generally preferred.

D. CHF1 Gene and Protein

The nucleotide and protein sequences of human and mouse CHF1 have been deposited in the GenBank/EBI Data Bank under GenBank accession numbers AF173901 and AF173902 respectively. The DNA sequences may be incorporated into an expression vector in which these sequences are operably linked to a promoter. The term “operably linked” refers to genetic elements that are joined in a manner that enables them to carry out their normal functions. For example, a gene is operably linked to a promoter when its transcription is under the control of the promoter and the transcript produced is correctly translated into the protein normally encoded by the gene.

The expression vectors can be used to transfect cardiac cells either in vitro or in vivo for the purpose of studying the effects of CHF1 expression and, ultimately as a therapeutic for the treatment of diseases associated with cardiac hypertrophy. Examples of eukaryotic promoters that may be used include the promoter of the mouse metallothionein I gene (Haymer, et al., J. Mol. Appl. Gen. 1:273 (1982)); the TK promoter of Herpes virus (McKnight, Cell 31:355-365 (1982)); the SV40 early promoter (Benoist, et al., Nature 290:304 (1981)), etc. A large number of plasmids suitable for use in eukaryotes have been described (Botstein, et al., Miami Winter Symp. 19:265 (1982); Broach, Cell 28:203 (1982); Bollon, et al., J. Cin. Hematol. Oncol. 10:39 (1980); Maniatis, in Cell Biology: A Comprehensive Treatise, vol. 3, Academic Press, M.Y. pp. 563-608 (1980)). In addition, sequences may be incorporated into DNA constructs designed for homologous recombination (see Capecchi, TIG 5:70 (1989); Mansour, et al., Nature 336:348 (1988); Thomas, et al., Cell 51:503 (1987); and Doetschman, et al., Nature 330:576 (1987)).

Once the vector or DNA sequence has been prepared, it may be introduced into an appropriate host cell by any suitable means of transfection (e.g., calcium phosphate precipitation or viral transfer). Standard methods of molecular biology may be used in preparing and manipulating sequences (see e.g., Sambrook et al., Molecular Cloning: A Laboratory Manual, 2nd ed. Cold Spring Harbor (1989)).

E. Assay Methods

CHF1 function may be used for the purpose of identifying compounds that inhibit cardiomyocyte hypertrophy. This can be accomplished by screening for compounds that suppress other phenotypic characteristics associated with mutations in the CHF1 gene. For example, the gridlock phenotype is caused by a loss of CHF1 activity and compounds that reverse this phenotype may be rapidly screened for in zebrafish (Peterson, et al., Nat. Biotechnol. 22:595-599 (2004)). Compounds identified as being active in this assay may then be further examined in a second assay that directly examines cardiac hypertrophy. For example, the compound may be examined in vitro for their effect on cardiac myocytes exposed to a hypertrophic agent such as phenylephrine. Agents identified as inhibiting cardiac cell proliferation have potential use in the treatment of diseases such as congestive heart failure or familial cardiac hypertrophy.

### EXAMPLES

### Example 1

### Suppression of Cardiac Hypertrophy by CHF1/Hey2

In the present example, we present evidence that overexpression of CHF1/Hey2 in the myocardium prevents phenylephrine-induced cardiac hypertrophy and expression of hypertrophy marker genes in vivo and in vitro and therefore functions as an antihypertrophic gene. Furthermore, we provide evidence that CHF1/Hey2 can directly block activation of a hypertrophy-associated gene, ANF, in reporter assays and that it interacts with GATA4, an important transcriptional activator of ANF and other hypertrophy-associated genes. The results suggest that CHF1/Hey2 can act as a suppressor of hypertrophy in vivo and in vitro, possibly through attenuation of a GATA4-dependent transcriptional program.

### I. Materials and Methods

Generation and characterization of transgenic mice that overexpress CHF1/Hey2 in myocardium: To examine the consequences of persistent overexpression of CHF1 in the developing and adult heart, we generated two independent FvB transgenic mouse lines expressing CHF1/Hey2 under the control of the ventricular regulatory myosin light chain (MLC2v) promoter. The minimal 250-bp MLC2v promoter has been shown to direct ventricular expression from embryonic day 9.0 through adulthood (O'Brien, et al., Proc. Nat'l Acad. Sci. USA 90:5157-5161 (1993)). We cloned a CHF1 cDNA consisting of nucleotides 76 to 1164 downstream of the minimal 250-bp MLC2v promoter and upstream of a bovine growth hormone polyadenylation signal before pronuclear injection. Pups were screened by Southern blotting and PCR of the MLC2v transgene unit. We chose this cDNA because it contains the entire protein coding sequence but will give rise to a smaller or “minigene” transcript (1.2 kb) compared with the endogenous transcript (˜2.6 kb), thereby allowing careful quantitation of transgene expression. Transgene copy number and expression level were determined using Southern blotting and Northern blotting, respectively. To assess expression of a variety of cardiac-specific genes, we isolated RNA from wild-type and transgenic hearts for semiquantitative RT-PCR as described previously (Sakata, et al., Proc. Nat'l Acad. Sci. USA 99:16197-16202 (2002)). Primer sequences are listed in Table 1. Echocardiographic assessments of left ventricular dimensions, wall thickness, and fractional shortening were essentially as described previously (Sakata, et al., Proc. Nat'l Acad. Sci. USA 99:16197-16202 (2002)).

Induction and assessment of phenylephrine-induced hypertrophy in vivo: Briefly, osmotic minipumps (Durect, Cupertino, Calif.) were loaded with phenylephrine or vehicle according to the manufacturer's instructions. Pumps were implanted subcutaneously between the scapula of 8-to 12-wk-old male transgenic and wild-type mice with the use of a sterile technique. Pumps infused either phenylephrine at a dose of 30 mg·kg⁻¹·day⁻¹ or vehicle for a 2-wk period. At the end of the study period, hearts were harvested for gravimetry, histology, RNA, protein, and cardiac myocyte isolation. Before death, echocardiographic measurement of ventricular wall thickness was performed as described previously (Sakata, et al., Proc. Nat'l Acad. Sci. USA 99:16197-16202 (2002)).

To assess effects of the transgene and phenylephrine on systemic blood pressure, we measured blood pressure using the tail-cuff method. Mice were trained by placement in the apparatus daily between the hours of 1:00 and 5:00 PM for 1 wk before any measurements were made. We subsequently measured blood pressure in wild-type and transgenic mice before, during, and after phenylephrine infusion. Blood pressure was measured on a daily basis beginning 1 wk after training and 1 wk before osmotic minipump implantation and then for the 2 wk following implantation.

Isolation and culture of mouse cardiac myocytes and assessment of cellular hypertrophy in vitro: Adult cardiac myocytes were isolated from male wild-type or transgenic mice after 2 wk of phenylephrine or vehicle treatment according to the Alliance for Cell Signaling protocol (Sambrano, et al., Nature 420:712-714 (2002)). Twenty-four hours after plating was completed, cells were immunostained with MF-20 monoclonal antibody against sarcomeric myosin (Developmental Studies Hybridoma Bank) and digitally photographed. Cell area was measured by quantitative morphometry of 50 consecutive cells for each condition with NIH Image software.

Neonatal cardiac myocytes were harvested using a modification of the method of Springhorn and Claycomb (Springhorn, et al., Biochem J. 258:73-78 (1989)). Hearts were removed and trimmed of atria and vascular tissue, and the remaining ventricular tissue from each heart was cut into several pieces. Tissue was incubated in 4 ml of collagenase type II solution (1 mg/ml) in HBSS and then transferred to a P60 dish and placed at 37° C. Cells were pipetted every 10 min until dispersed (up to 30-40 min) and then filtered with a 70 μm nylon cell strainer (FALCON 35-2350) on a 50-ml tube to remove tissue debris. Collagenase was neutralized in the filtrate by the addition of DMEM-20% FCS. Cells were collected by centrifugation at 700 rpm for 5 min, resuspended in 10 ml of DMEM-20% FCS, and incubated on a P100 dish at 37° C. for 1-2 h to remove fibroblasts. The nonadherent cells were collected by centrifugation of the culture medium at 700 rpm for 5 min and resuspended in 10.5 ml of DMEM-20% FCS, and the number of cells was quantified by Coulter counting. Cells were seeded onto fibronectin coated dishes at the density of 1×10⁵ cells/well in a 24-well plate for hypertrophy assays and 3-5×10⁶ cells onto P60 dishes for other purposes. Ara-C (20 μM) was included in the culture medium to inhibit proliferation of contaminating fibroblasts.

For hypertrophy assays, cells were allowed to attach for 24 h in DMEM-20% FCS and then changed to serum-free DMEM (catalog no. 11995-065, with penicillin streptomycin, GIBCO) containing 10 μg/ml insulin [1.744 mom stock as 1,000× (10 mg/ml); catalog no. I-1882, Sigma], 10 μg/ml transferring (catalog no. T-8158, Sigma), 1 mg/ml BSA, and 20 μM Ara-C. After 24 h in serum-free medium, cells were stimulated with phenylephrine (20 μM) and timolol (2 μM) or vehicle for 48 h. Cells were immunostained for sarcomeric actin with MF-20 monoclonal antibody (Developmental Studies Hybridism Bank), and cell size was quantified using morphometry as described above.

Transfection, coimmunoprecipitation assays, and Western blotting: For transfection assays, neonatal cardiac myocytes were cultured in DMEM-10% FCS and transfected with FuGENE 6 according to the manufacturer's protocol (Roche). Cells were transfected with plasmids driving expression of CHF1/Hey2 (Chin, et al., J. Biol. Chem. 275: 6381-6387 (2000)) or GATA4 (Svensson, Nat. Genet. 25:353-356 (2000)) under the control of the human cytomegalovirus promoter, in conjunction with an ANF-luciferase reporter construct (Sprenkle, et al., Circ. Res. 77:1060-1069 (1995)). An expression vector containing the CMV promoter with no insert (pcDNA3; Promega, Madison, Wis.) was used to normalize the total DNA for each transfection. Cells were harvested for luciferase assays after 48 h, and firefly luciferase activity driven by the ANF promoter (Sprenkle, et al., Circ. Res. 77:1060-1069 (1995)) was normalized to Renilla luciferase activity according the manufacturer's protocol (Promega).

For coimmunoprecipitation, COS-7 cells were transfected with plasmids expressing FLAG-tagged CHF1/Hey2 and myc-tagged GATA4 by using FuGENE 6 as described above. Lysates were made, and immunoprecipitation with 9E10 monoclonal antibody against the myc tag or M2 antibody against the FLAG tag followed by Western blotting was performed essentially as described previously (Sun, et al., J. Biol. Chem. 276:18591-18596 (2001)), except that SDS was omitted from the lysis buffer.

Electrophoretic mobility shift assays: GATA4 and CHF1/Hey2 proteins were synthesized from plasmid templates by in vitro transcription and translation with a commercially available kit according to the manufacturer's instructions (Promega). Oligonucleotides containing the binding site for GATA4 within the mouse ANF promoter were synthesized, annealed, and labeled using standard methods. The primer sequences are listed in Table 1. Binding conditions and electrophoresis for CHF1/Hey2 and GATA4 were similar to those previously described (Chin, et al., J. Biol. Chem. 275: 6381-6387 (2000); Sun, et al., J. Biol. Chem. 276:18591-18596 (2001)), except that the binding buffer consisted of 10 mM Tris, pH 7.4, 50 mM NaCl, 1 mM dithiothreitol, 1 mM EDTA, and 4% glycerol, and electrophoresis was performed with 1×Tris-borate-EDTA at room temperature.

TABLE 1
Primer Sequences

| | | |
|---|---|---|
| ANF_L | 5′-GTC AAT CCT ACC CCC GAA GCA GCT-3′ | (SEQ ID NO: 1) |
| ANF_R | 5′-CAG CAT GGG CTC CTT CTC CA-3′ | (SEQ ID NO: 2) |
| BNP-S | 5′-ATG GAT CTC CTG AAG GTG CT-3′ | (SEQ ID NO: 3) |
| BNP-AS | 5′-AAG AGG GCA GAT CTA TCG GA-3′ | (SEQ ID NO: 4) |
| CHF1-S | 5′-GAC AAC TAC CTC TCA GAT TAT GGC-3′ | (SEQ ID NO: 5) |
| CHF1-AS | 5′-CGG GAG CAT GGG AAA AGC-3′ | (SEQ ID NO: 6) |
| CHF2-S | 5′-CAT GAA GAG AGC TCA CC-3′ | (SEQ ID NO: 7) |
| CHF2-AS | 5′-AAT GTG TCC GAG GCC AC-3′ | (SEQ ID NO: 8) |
| CHF3-S | 5′-ATG GAC CCA TCG ATG TGG-3′ | (SEQ ID NO: 9) |
| CHF3-AS | 5′-AAA GGC CAG GGC ACT GG-3′ | (SEQ ID NO: 10) |
| HPRT-S | 5′-CTC GAA GTG TTG GAT ACA GG-3′ | (SEQ ID NO: 11) |
| HPRT-AS | 5′-TGG CCT ATA GGC TCA TAG TG-3′ | (SEQ ID NO: 12) |
| αMHC-S | 5′-CTG CTG CAG AGG TTA TTC CTC G-3′ | (SEQ ID NO: 13) |
| αMHC-AS | 5′-GGA AGA GTG AGC GGC GCA TCA AGG-3′ | (SEQ ID NO: 14) |
| βMHC-S | 5′-TGC AAA GGC TCC AGG TCT GAG GGC-3′ | (SEQ ID NO: 15) |
| βMHC-AS | 5′-GCC AAC ACC AAC CTG TCC AAG TTC-3′ | (SEQ ID NO: 16) |
| MLC2v-S | 5′-CTCAGTCCTTCTCTTCTCCG-3′ | (SEQ ID NO: 17) |
| MLC2v-AS | 5′-TGTTCCTCACGATGTTTGGG-3′ | (SEQ ID NO: 18) |
| Nkx2.5/Csx-S | 5′-GCAGAAGGCAGTGGAGCTGGACAAAGCC-3′ | (SEQ ID NO: 19) |
| Nkx2.5/Csx-AS | 5′-TTGCACTTGTAGCGACGGTTCTGGAACCAG-3′ | (SEQ ID NO: 20) |
| MEF2c-S | 5′-GCCCTGAGTCTGAGGACAAG-3′ | (SEQ ID NO: 21) |
| MEF2c-AS | 5′-ATCGTGTTCTTGCTGCCAG-3′ | (SEQ ID NO: 22) |
| GATA4_L | 5′-CAC TAT GGG CAC AGC AGC TCC-3′ | (SEQ ID NO: 23) |
| GATA4_R | 5′-TTG CAG CTG GCC TGC GAT GTC-3′ | (SEQ ID NO: 24) |
| GAPDH_L | 5′-GCT TCA CCA CCT TCT TG-3′ | (SEQ ID NO: 25) |
| GAPDH_R | 5′-TCA CCA TCT TCC AGG AG-3′ | (SEQ ID NO: 26) |
| ANF GATA4 | 5′-CTT TTA AAG TTA TCA GCT CAG CGA AGC-3′ | (SEQ ID NO: 27) |
| BINDING SITE 1 | | |
| ANF GATA4 | 5′-GCT TCG CTG GAC TGA TAA CTT TAA AAG-3′ | (SEQ ID NO: 28) |
| BINDING SITE 2 | | |
| ANF GATA4 | 5′-CTT TTA AAG TTA AGA GCT CAG CGA AGC-3′ | (SEQ ID NO: 29) |
| BINDING SITE 1 | | |
| MUTANT | | |
| ANF GATA4 | 5′-GCT TCG CTG GAC TCT TAA CTT TAA AAG-3′ | (SEQ ID NO: 30) |
| BINDING SITE 2 | | |
| MUTANT | | |

### II. Results

Generation and characterization of CHF1/Hey2 transgenic mice: Our group has previously reported that CHF1/Hey2 is expressed at high levels in the developing ventricle from approximately embryonic day 8.5 until birth and is then expressed at lower levels in the postnatal heart (Chin, et al., J. Biol. Chem. 275:6381-6387 (2000)). Furthermore, our group has reported that targeted disruption of the gene for CHF1/Hey2 leads to cardiomyopathy (Sakata, et al., Proc. Nat'l Acad. Sci. USA 99:16197-16202 (2002)). To examine the consequences of persistent overexpression of CHF1/Hey2 in the developing and adult heart, we generated transgenic mice expressing CHF1/Hey2 under the control of the MLC2v promoter. Two independent FvB transgenic lines that express the cDNA were generated. The transgene copy number for both lines was between 1 and 5. It was found that the transgene mRNA was expressed at high levels in all transgenic lines and was specific to myocardium, although there was some ectopic atrial expression that was not expected.

Careful embryonic and anatomic pathological analysis of transgenic mice revealed no alterations in cardiovascular development. Furthermore, transgenic mice were indistinguishable from wild-type littermates in terms of survival to weaning or adulthood (up to 52 wk). Serial echo-cardiograms (up to 40 wk) showed no significant differences in wall thickness, left ventricular dimensions, heart rate, or fractional shortening. To assess for baseline alterations in cardiac gene expression, we analyzed RNA from wild-type and transgenic hearts by using RT-PCR. It was found that transgenic overexpression of CHF1/Hey2 did not alter basal expression of the related transcription factors CHF2/Hey1 and CHF3/HeyL, and it did not alter basal expression of the cardiac-specific genes Nkx2.5, GATA4, MLC2v, myocyte enhancer factor 2c (MEF2c), α-MHC, or β-MHC. Similar results were seen with both transgenic lines. Together, these results suggest that overexpression of CHF1/Hey2 in the myocardium has no obvious effects on normal cardiovascular development or function.

CHF1/Hey2 transgenic mice are resistant to phenylephrine-induced hypertrophy and do not demonstrate induction of hypertrophy-associated markers: As mentioned, the absence of CHF1/Hey2 leads to cardiomyopathy (Sakata, et al., Proc. Nat'l Acad. Sci. USA 99:16197-16202 (2002)), and overexpression of CHF1/Hey2 does not lead to any obvious developmental, anatomical, or functional abnormalities at baseline and does not have any effect on overall viability and mortality. To test the hypothesis that CHF1/Hey2 may prevent the development of hypertrophy, we challenged the transgenic mice with phenylephrine infusion. Phenylephrine, an α-adrenergic agonist, is a commonly used hypertrophic agent, both in vitro on isolated cardiac myocytes and in vivo through the use of osmotic minipump infusion.

Briefly, osmotic minipumps were implanted into 8-wk-old transgenic and wild-type mice subcutaneously. Pumps infused either phenylephrine at a dose of 30 mg·kg⁻¹·day⁻¹ or vehicle for a 2-wk period. At the end of the study period, hearts were harvested for gravimetry, histology, RNA, protein, and cardiac myocyte isolation. It was found that histological sections from wild-type mice stained with hematoxylin and eosin show the expected increase in wall thickening after phenylephrine treatment. Transgenic mice, in contrast, did not show any obvious increase in wall thickness or heart size. No differences were seen in fibrosis or apoptosis as measured using Masson trichrome staining and TdT-mediated dUTP nick end labeling staining. To quantify the degree of hypertrophy, we calculated the ventricular weight-to-body weight ratio (VW/BW) for wild-type and transgenic mice before and after phenylephrine treatment. It was found that the VW/BW increased for wild-type mice after phenylephrine treatment but did not change significantly in the transgenic mice. Echocardiographic assessment of wall thickness before and after treatment also showed no significant increase in transgenic mice treated with phenylephrine compared with wild-type mice. Left ventricular function was preserved before and after treatment for both groups. Although this finding is somewhat surprising, it may reflect the limited dose and timing of phenylephrine infusion such that hypertrophy develops, but progression to left ventricular dysfunction has not yet occurred.

Prolonged infusion would be expected to cause hypertrophy and decrease left ventricular function in wild-type mice. Similar results were seen in both transgenic lines. ANF, BNP, and β-MHC mRNAs were induced in wild-type but not transgenic animals treated with phenylephrine, demonstrating that expression of these marker genes correlates with the development of hypertrophy seen in vivo. CHF1/Hey2 and GATA4 mRNAs were unaffected in both wild-type and transgenic animals. These results were quantified using densitometry. Interestingly, the expression of endogenous CHF1/Hey2 was reproducibly decreased with phenylephrine treatment, consistent with its potential function as an antihypertrophic gene.

Cardiac myocytes from transgenic mice are resistant to phenylephrine-induced hypertrophy in vivo and in vitro: To determine that the effect of CHF1/Hey2 on cardiac hypertrophy is a primary effect on the monocyte rather than caused by effects on other cells, we initially isolated cardiac myocytes from adult wild-type or transgenic mice after 2 weeks of phenylephrine or vehicle treatment. It was found that two dimensional cell area was not significantly different for wild-type cells and transgenic cells treated with vehicle. With phenylephrine treatment, wild-type cells demonstrated increased size compared with vehicle controls but transgenic cells did not. Three independent preparations were analyzed.

We also isolated neonatal cardiac myocytes from wild-type and transgenic animals and treated them with phenylephrine in vitro. Although others Deng, et al., Circ. Res. 87:781-788 (2000)) have reported that mouse neonatal myocytes do not develop hypertrophy in response to phenylephrine in vitro, in our hands, wild-type monocytes did develop hypertrophy in response to phenylephrine. The reasons for this discrepancy are unclear but may be related to the different protease treatments used in the isolation protocols. Transgenic myocytes, in contrast, did not develop hypertrophy in response to phenylephrine in vitro. Three independent preparations were analyzed. These findings are consistent with the failure of the transgenic animals to develop increased VW/BW and demonstrate that the effect is mediated at the myocyte level.

Cardiac myocytes from transgenic mice do not demonstrate induction of hypertrophy markers after phenylephrine treatment in vitro: To assess whether induction of a hypertrophic transcriptional program accompanies the observed changes in myocyte cell size, we isolated RNA from cultured neonatal myocytes treated with phenylephrine. Semiquantitative RTPCR of ANF, BNP, and β-MHC mRNAs from cultured wild-type and transgenic neonatal myocytes treated with either vehicle or phenylephrine demonstrated that ANF, BNP, and β-MHC expression is induced in wild-type myocytes by phenylephrine, as expected. Transgenic myocytes, in contrast, did not demonstrate induction of these mRNAs. These findings indicate that the resistance to hypertrophy observed in the transgenic myocytes is accompanied by absence of induction of a hypertrophic transcriptional program. Endogenous CHF1/Hey2 mRNA was reproducibly decreased, whereas transgenic CHF1/Hey2 and GATA4 mRNAs were not affected by phenylephrine treatment in vitro, which mirrors their responses to phenylephrine infusion in vivo.

CHF1/Hey2 transgenic mice have blood pressures comparable to those of wild-type mice before and during phenylephrine infusion: To assess whether the myocardial CHF1/Hey2 transgene may affect systemic blood pressure, we carefully measured blood pressure before, during, and after phenylephrine infusion. It was found that blood pressure was not significantly different in wild-type and transgenic mice before and during phenylephrine treatment. These findings indicate that transgenic expression of CHF1/Hey2 in the myocardium does not alter baseline blood pressure and does not affect the blood pressure response to phenylephrine at the doses used. Consequently, the hypertrophy seen in wild-type myocytes is likely due to a primary effect on the myocardium.

CHF1/Hey2 suppresses GATA4-dependent transcription of ANF promoter in primary neonatal cardiac myocytes, interferes with GATA4 binding to DNA, and interacts directly with GATA4: GATA4 previously has been shown to activate hypertrophy-associated genes in vivo and in vitro (Liang, et al., J. Biol. Chem. 276:30245-30253 (2001)). ANF expression is commonly induced by hypertrophic stimuli, and its promoter is GATA4 dependent and often used as a reporter for induction of hypertrophy (Sprenkle, et al., Circ. Res. 77:1060-1069 (1995)). GATA4 mutations also have been associated with septal defects (Garg , et al., Nature 424:443-447 (2003)), which we also have observed in CHF1/Hey2 knockout mice (Sakata, et al., Proc. Nat'l Acad. Sci. USA 99:16197-16202 (2002)). Furthermore, deletion of the GATA4-interacting repressor protein gene, FOG2, gives rise to a spectrum of anomalies reminiscent of those seen in the CHF1/Hey2 null mice, such as tricuspid stenosis and septal defects (Svensson, Nat. Genet. 25:353-356 (2000); Tevosian, et al., Cell 101:729-739 (2000)). Given that CHF1/Hey2 prevents the development of hypertrophy in vivo and in vitro and blocks induction of ANF mRNA in cultured cardiac myocytes, we assessed whether CHF1/Hey2 can directly inhibit GATA4-dependent activation of ANF in reporter assays. The results obtained suggest that GATA4 can activate the promoter, as expected, and that cotransfection of CHF1/Hey2 can block this activation.

To determine the mechanism by which CHF1/Hey2 blocks GATA4-dependent activation of the ANF promoter, we tested the hypothesis that CHF1/Hey2 may interfere with the binding of GATA4 to its specific binding sequence within the promoter. GATA4 is known to bind specifically to the ANF promoter and activate it in conjunction with the cardiac-specific factor Nkx2.5 (Durocher, et al., EMBO J. 16:5687-5696 (1997); Lee, et al., Mol. Cell Biol. 18:3120-3129 (1998)). To test this hypothesis, we performed electrophoretic mobility shift assays essentially as described previously (Chin, et al., J. Biol. Chem. 275: 6381-6387 (2000); Sun, et al., J. Biol. Chem. 276:18591-18596 (2001)), with minor modifications as described above under “Materials and Methods.” The results obtained suggest that GATA4 binds to the ANF promoter oligonucleotide and that CHF1/Hey2, when added to GATA4, inhibits formation of the GATA4 complex in a dose-dependent fashion. The specificity of the GATA4 complex could be further demonstrated by competition with an unlabeled ANF promoter oligonucleotide and by failure of a mutant ANF promoter oligonucleotide to compete for binding. Quantification by densitometry confirmed these findings. The results demonstrate that CHF1/Hey2 directly inhibits binding of GATA4 to its recognition sequence within the ANF promoter.

To examine further the potential mechanism by which CHF1/Hey2 regulates GATA4 activity, we tested the hypothesis that CHF1/Hey2 may interact directly with GATA4. CHF1/Hey2 and its relatives have been shown to interact with several important transcriptional regulatory proteins, such as the arylhydrocarbon receptor nuclear translocator ARNT (Chin, et al., J. Biol. Chem. 275: 6381-6387 (2000)), MyoD (Sun, et al., J. Biol. Chem. 276:18591-18596 (2001)), mSin3, and HES proteins (Iso, et al., Mol. Cell Biol. 21:6080-6089 (2001)). To assess the ability of GATA4 to interact with CHF1/Hey2, we coexpressed myc-tagged GATA4 and FLAG-tagged CHF1/Hey2 in COS-7 cells. It was found that immunoprecipitation of myc-tagged GATA4 coimmunoprecipitated FLAG-tagged CHF1/Hey2. Similarly, immunoprecipitation of FLAG-tagged CHF1/Hey2 coimmunoprecipitated myc-tagged GATA4. Our findings indicate that CHF1/Hey2 interacts directly with GATA4 and forms an inactive complex that prevents DNA binding and transcriptional activation.

### III. Discussion

We have found that the bHLH transcriptional repressor CHF1/Hey2 functions as an antihypertrophic protein, both in vitro and in vivo. Furthermore, we have found that CHF1/Hey2 can directly inhibit activation of a hypertrophy-associated gene, ANF, through a direct inhibitory interaction with the transcription factor GATA4 that interferes with DNA binding. Our findings imply that CHF1/Hey2 functions as an antihypertrophic protein by inhibiting the activation of a GATA4dependent hypertrophy transcriptional program.

The interaction between CHF1/Hey2 and GATA4 was independently described in a recent publication, and a similar effect on ANF promoter activity in vitro was demonstrated (Kathiriya, et al., J. Biol. Chem. 279:54937-54943 (2004)). Our findings, however, provide additional evidence that this interaction is relevant to the hypertrophic response in vivo. An interaction between the related factor CHF2/Hey1 (also known as HERP2) and the erythroid factor GATA1 was recently reported to inhibit hematopoiesis in vitro (Elagib, et al., Mol. Cell Biol. 24:7779-7794 (2004)). A similar report demonstrating an interaction between HES-1 and GATA1 resulting in decreased hematopoiesis in vitro was also published recently (Ishiko, et al., J. Biol. Chem. 280:4929-4939 (2005)). These findings suggest that interactions between hairy-related transcription factors and GATA-related transcription factors to regulate important biological processes is likely to be a general phenomenon.

Analysis of our data on ANF reporter gene activity, GATA4 DNA binding, and coimmunoprecipitation suggests that the effect of CHF1/Hey2 on GATA4 DNA binding is relatively weak compared with the effect on reporter gene transcription and in relation to the relative robustness of the protein-protein interaction. A previous report (Kathiriya, et al., J. Biol. Chem. 279:54937-54943 (2004)) presents data that the CHF1/Hey2 (also known as HRT2) interaction with GATA4 does not prevent DNA binding, although different binding conditions were used. The relatively weak effect of CHF1/Hey2 in vitro compared with inside cells may be due to an additional effect of CHF1/Hey2 on GATA4 interactions with coactivators such as p300 or corepressors such as FOG2, or to an effect on GATA4 posttranslational modification such as phosphorylation at serine 105 (Liang, et al., Mol. Cell Biol. 21:7460-7469 (2001)).

To date, relatively few antihypertrophic transcriptional regulatory genes have been described, and little is known about the mechanisms by which the majority of these genes function. An antihypertrophic transcriptional program has been postulated to explain the phenotype of mice overexpressing the homeodomain only protein, HOP (Chen, et al., Cell 110:713-723 (2002)). HOP has been shown to inhibit SRF-dependent transcription in vitro by preventing the binding of SRF to its target CArG boxes, and targeted disruption of the gene for HOP in mice leads primarily to embryonic death due to abnormalities in cardiac development (Chen, et al., Cell 110:713-723 (2002); Shin, et al., Cell 110:725-735 (2002)). Transgenic mice that overexpress HOP in the myocardium, in contrast, develop spontaneous cardiac hypertrophy through recruitment of class I inhibitory histone deacetylases (HDACs) and inhibition of an SRF-dependent antihypertrophic program (Kook, et al., J. Clin. Invest. 112:863-871 (2003)). The identities of these putative downstream antihypertrophic genes remain generally unknown, although there are multiple reports of individual transcriptional regulatory genes exerting an antihypertrophic effect, such as HDAC9 (Zhang, et al., Cell 110:479-488 (2002)) and the RNA helicase CHAMP (Liu, et al., Proc. Nat'l Acad. Sci. USA 99:2043-2048 (2002)). The relationship of CHF1/Hey2 to these other proteins associated with suppression of hypertrophy remains under investigation, but it is intriguing to speculate that CHF1/Hey2 may act as an antihypertrophic protein downstream of some of these antihypertrophic molecules.

### Example 2

### Suppression of Cardiomyocyte Hypertrophy by GS4012

The effect of GS4012 on the hypertrophy of mouse cardiomyoctes was studied in vitro. Cells were incubated under four different conditions: A: vehicle alone (the vehicle is used to dissolve GS4012 and is dimethysulfoxide (DMSO)); B: vehicle+fetal calf serum (FCS; the fetal calf serum serves as an agent that induces cardiomyocyte hypertrophy); C: vehicle+2.5 μg GS4012; and D: vehicle+2.5 μg GS4012+FCS. Cell hypertrophy was determined by the incorporation of ³H-leucine. Tables 2 and 3 below show the results that were obtained (expressed as counts per minute).

It can be seen that, as expected, FCS induces cardiomyocyte proliferation (compare counts per minute (cpm) for cells incubated in DMSO alone with cpm for cells incubated in DMSO containing FCS). This induction of proliferation did not occur when GS4012 was present (compare cpm for vehicle+GS4012 with cpm for vehicle+GS4012+FCS.

TABLE 2
Effect of GS4012 on Cardiomyocyte Hypertrophy

| Test No. | DMSO | DMSO + FCS | Test No. | GS4012 | GS4012 + FCS |
|---|---|---|---|---|---|
| 1 | 460 | 295.67 | 1 | 183 | 148.33 |
| 2 | 508 | 782.67 | 2 | 251.67 | 118 |
| 3 | 418 | 820.33 | 3 | 208.33 | 145 |
| 4 | 355 | 818 | 4 | 442.33 | 277 |
| 5 | 384.67 | 536 | 5 | 446.67 | 204.33 |
| 6 | 449.33 | 455.33 | 6 | 393 | 496.33 |
| 7 | 660 | 702 | 7 | 297 | 630.33 |
| 8 | 512 | 854.33 | 8 | 503 | 382.33 |
| 9 | 635 | 887.67 | 9 | 591 | 454 |
| 10 | 217 | 805.33 | 10 | 377 | 356.67 |
| 11 | 541 | 610.67 | 11 | 439.33 | 182.67 |
| 12 | 560 | 887.67 | 12 | 639.67 | 692 |
| mean | 475 | 704.639167 | mean | 397.6667 | 340.5825 |
| SE | | | | | |
| ttest DMSO | 0.0023759 | ttest GS4012 vs | 0.4239689 | | |
| vs | | GS4012 + FCS | | | |
| DMSO + FCS | | ttest | | | |
| ttest DMSO | 0.1713084 | DMSO + FCS vs | 0.0001309 | | |
| vs GS4012 | | GS4012 + FCS | | | |

TABLE 3
Effect of GS4012 on Cardiomyocyte Hypertrophy

| | | DMSO, | GS4012 | GS4012 |
|---|---|---|---|---|
| Test No. | WT DMSO | FCS | 2.5 μg/ml | FCS |
| 1 | 187.33 | 765 | 245.33 | 132.67 |
| 2 | 278.33 | 680 | 346 | 331.33 |
| 3 | 355.33 | 585.67 | 381.33 | 438.33 |
| 4 | 263 | 557.33 | 361.33 | 680.67 |
| 5 | 378 | 767 | 396 | 418.67 |
| 6 | 247.67 | 570 | 253.67 | 498 |
| 7 | 403 | 800 | 315 | 439.67 |
| 8 | 458 | 513.33 | 336.33 | 419 |
| mean | 324.7075 | 654.79125 | 329.37375 | 419.7925 |
| SE | 31.34185106 | 34.94740455 | 17.2745586 | 47.89732493 |
| mean | 324.7 | 654.8 | 329.37 | 419.8 |
| SE | 31.34185106 | 34.94740455 | 17.2745586 | 47.89732493 |
| t test | | | | |
| t test DMSO vs DMSO | 1.70645E−05 | | t test GS4012 | 0.152565377 |
| FCS | | | vs GS4012 | |
| | | | FCS | |
| t test DMSO FCS vs | 0.004031359 | | | |
| GS4012 FCS | | | | |

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

1. A pharmaceutical composition in unit dose form comprising a compound that increases the activity of the CHF1 gene product in cardiomyocytes, a pharmaceutically acceptable vehicle and one or more excipients.

2. The pharmaceutical composition of claim 1, wherein said compound has a structure according to formula I: 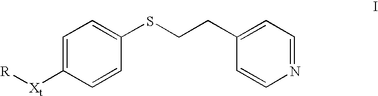
where X is O, N, C or S and t is 0 or 1;
R is selected from the group consisting of: H; a straight or branched C₁-C₆ alkyl; a straight or branched C₂-C₆ alkenyl; —CH₂)ₐ—C(O)—R₁; and —(CH₂)ₐ—NR₂R₃, where R₁-R₃ are each independently selected from: H; halogen; and C₁-C₃ alkyl; and a is an integer from 0 to 3;
and wherein the phenyl or pyridine rings in formula I may optionally be substituted by one or more substituents selected from the group consisting of: halogen; OH; and C₁-C₃ alkyl.

3. The pharmaceutical composition of claim 2, wherein said compound is selected from the group consisting of: (4-(2-(4-Methoxyphenylsulfanyl)ethyl)pyridine; 4-[2-(4-pyridinyl)-ethylsulfanyl]phenoxy acetic acid; and 4-[2-(4-dimethylaminoethoxy-phenylsulfanyl)-ethyl]pyridine.

4. A method of preventing the hypertrophy of cardiomyocytes in a patient, comprising administering to said patient a therapeutically effective amount of a compound that increases the activity of the CHF1 gene product in cardiomyocytes.

5. The method of claim 4, wherein said compound has a structure according to formula I: 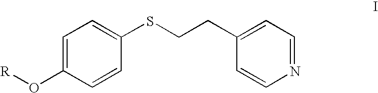
where R is selected from the group consisting of: H; a straight or branched C₁-C₆ alkyl; a straight or branched C₂-C₆ alkenyl; —CH₂)ₐ—C(O)—R₁; and —CH₂)ₐ—NR₂R₃, where R₁-R₃ are each independently selected from: H; halogen; and C₁-C₃ alkyl; and a is an integer from 0 to 3;
and wherein the phenyl or pyridine rings in formula I may optionally be substituted by one or more substituents selected from the group consisting of: halogen; OH; and C₁-C₃ alkyl.

6. The method of claim 4, wherein said compound is selected from the group consisting of: (4-(2-(4-Methoxyphenylsulfanyl)ethyl)pyridine; 4-[2-(4-pyridinyl)-ethylsulfanyl]phenoxy acetic acid; and 4-[2-(4-dimethylaminoethoxy-phenylsulfanyl)-ethyl]pyridine.

7. The method of claim 4, wherein said patient has congestive heart failure.

8. The method of claim 4, wherein said patient has familial cardiac hypertrophy.

9. A compound of formula I: 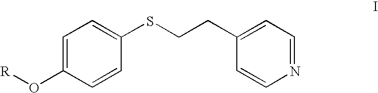
where R is selected from the group consisting of: H; a straight or branched C₃-C₆ alkyl; a straight or branched C₂-C₆ alkenyl; —(CH₂)ₐ—C(O)—R₁; and —(CH₂)ₐ—NR₂R₃, where R₁-R₃ are each independently selected from: H; halogen; and C₁-C₃ alkyl; and a is an integer from 0 to 3;
and wherein the phenyl or pyridine rings in formula I may optionally be substituted by one or more substituents selected from the group consisting of: halogen; OH; and C₁-C₃ alkyl.

10. The compound of claims 9, wherein R is —(CH₂)ₐ—C(O)—R₁.

11. The compound of claims 9, wherein R is —(CH₂)ₐ—NR₂R₃.

12. The compound of claim 9, wherein the phenyl and pyridine rings in the compound of formula I are each substituted at no more than two positions.

13. The compound of claim 9, wherein the phenyl and pyridine rings in the compound of formula I are unsubstituted.

14. (canceled)

15. The compound of claim 9, wherein said compound is either: 4-[2-(4-pyridinyl)-ethylsulfanyl]phenoxy acetic acid; or 4-[2-(4-dimethylaminoethoxy-phenylsulfanyl)-ethyl]pyridine.

16. A method of determining if a test compound is useful in the treatment of a condition characterized by cardiomyocyte hypertrophy, comprising:
a) assaying said test compound for its ability to suppress a phenotypic characteristic caused by a loss of CHF1 expression; and
b) if the compound is found to be effective in the assay of paragraph a), further testing said compound in an assay of cardiomyocyte hypertrophy.

17. The method of claim 16, wherein said ability to suppress a phenotypic characteristic caused by a loss of CHF1expression is determined using zebrafish.

18. The method of claim 17, wherein said phenotypic characteristic is the gridlock phenotype.

19. A method of inhibiting the proliferation of cardiomyocytes, comprising transfecting said cardiomyocytes with a vector comprising a promoter active in said cardiomyocytes operably linked to a sequence coding for the human or mouse CHF1 transcription factor.
